(19) United States
(12) Patent Application Publication
(10) Pub. No.: US 2011/0136211 A1
(43) Pub. Date: Jun. 9, 2011

(54) Mist or Spray Microorganism Cultivation and Related Methods

(76) Inventor: Qi Zhong, San Diego, CA (US)

(21) Appl. No.: 12/633,775

(22) Filed: Dec. 8, 2009

(51) Int. Cl. C12N 1/20; C12M 1/00; C12N 1/00

(52) U.S. Cl. 435/252.8; 435/283.1; 435/243

(57) ABSTRACT

Disclosed is an apparatus for culturing microorganisms and methods for using the same.

### CROSS-REFERENCE TO RELATED APPLICATIONS

N/A

### STATEMENT REGARDING FEDERALLY SPONSORED RESEARCH OR DEVELOPMENT

N/A

### BACKGROUND OF THE INVENTION

1. Field of Invention

The present invention is in the field of microorganism cultivation.

2. Background of the Invention

Microorganisms are frequently exploited in scientific, environmental, industrial, medical, medicinal, educational, military, nutritional, waste-management, hygienic, bio-fuel, green technology, and biotechnology applications. Frequently, microorganism exploitation depends on an ability to cultivate a microorganism culture. Successful microorganism culturing can depend, in part, on: (1) the delivery of nutrients and/or light to the culturing medium; (2) the removal of metabolic products from the culturing medium; and, (3) the manipulation or control of culture temperature. The present application discloses preferable embodiments of an apparatus and associated methods for improved delivery of nutrients and/or light to a microorganism culture.

Frequently, gaseous nutrients for cultivation must be provided to microorganisms within a liquid culturing medium. For example: aerobic microorganisms in a liquid medium depend on access to oxygen (O₂) for development and growth; and, photoautotrophs in a liquid medium require carbon dioxide (CO₂), in addition to oxygen, in order to grow and develop. Accordingly, there is a need for apparatus and methods for delivering gaseous nutrients to microorganisms in a liquid culturing medium.

Ordinarily, the provision of gaseous nutrients to microorganisms in a liquid culturing medium has been inefficient in circumstances where the gas and medium are merely placed in the same environment since the medium naturally settles into a pool underneath the gas whereby nutrient transfer can only occur at the upper liquid surface. Inefficiencies arise because gaseous nutrients are not provided to microorganisms at the bottom of the culturing medium pool without altering the natural interaction of the gas and medium. As a result, only a fraction of the microorganism culture receives the nutrients essential for cultivation.

To address this problem, some have attempted to (1) stir the culture medium, (2) bubble-up gaseous nutrients through the culturing medium, (3) a combination of the stirring and bubbling, or (4) dissolving gaseous nutrients into a solution and providing the same to immobilized microorganisms. For example: U.S. Pat. No. 4,654,308 (issued Mar. 31, 1987) discloses a tray-tower apparatus wherein a liquid culturing medium flows laterally across a tray and downward from tray-to-tray at the tray sides while a gaseous nutrient moves upward through the flowing medium via apertures in each tray; U.S. Pat. No. 4,952,511 (issued Aug. 28, 1990) discloses a chamber filled with a liquid culturing medium wherein gaseous nutrients are bubbled through the chamber; U.S. Pat. No. 5,057,428 (issued Oct. 15, 1991) discloses a tank with mesh piping wherein gaseous nutrients are pumped through the piping for release into a liquid culturing medium within the tank; U.S. Pat. No. 5,075,234 (issued Dec. 24, 1991) discloses a tank which stirs a liquid culturing medium while air is being provided to the medium through apertures at pre-defined locations in the tank wall; U.S. Pat. No. 5,587,298 (issued Dec. 24, 1996) discloses a tank with a helical screw for mixing a liquid culture medium in the presence of a gaseous nutrient outlet; U.S. Pat. No. 5,616,493 (issued Apr. 1, 1997) discloses a plug flow reactor for mixing (“vigorous agitation”) gaseous nutrients with a liquid culturing medium to create a foam (i.e., a “liquid medium surrounding a discontinuous gaseous phase” (col. 4:66-67)); U.S. Pat. No. 5,660,977 (issued Aug. 26, 1997) discloses a tank that circulates a liquid culturing medium while bubbling gaseous nutrients therethrough; U.S. Pat. No. 6,245,555 (issued Jun. 12, 2001) discloses a tank with an aerator for bubbling gas through a liquid culturing medium; U.S. Pat. No. 6,395,175 (issued May 28, 2002) discloses an apparatus for improved bubbling of gaseous nutrients through a liquid culturing medium; U.S. Pat. No. 6,432,698 (issued Aug. 13, 2002) discloses a disposable tank wherein gaseous nutrients may be bubbled through a liquid culturing medium; U.S. Pat. No. 6,509,118 (issued Jan. 21, 2003) discloses a transparent tank wherein gaseous nutrients may be bubbled through a liquid culturing medium; U.S. Pat. No. 6,916,652 (issued Jul. 12, 2005) discloses an apparatus wherein “biocatalysts are substantially immobilized contained, suspended and or incubated in a chamber;” U.S. Pat. No. 7,232,664 (issued Jun. 19, 2007) discloses the formation of smaller bubbles of gaseous nutrients within a liquid culturing medium to increase nutrient transfer rates; U.S. Pat. No. 7,485,454 (issued Feb. 3, 2009) discloses a reaction chamber wherein cells are immobilized and dissolved gaseous nutrients are provided thereto; U.S. Pat. No. 7,507,579 (issued Mar. 24, 2009) discloses a small chamber with a membrane separating oxygenated water from a culturing medium, wherein the oxygen in the water will move through the membrane into the culturing medium via osmosis; U.S. Pat. No. 7,521,203 (issued Apr. 21, 2009) discloses an example wherein a liquid culturing medium is provided with oxygen via stirring and bubbling (“agitation” and “air flow”); US Pub. Pat. App. No. 20090263877 (published Oct. 22, 2009) discloses a loop reactor wherein oxygen is introduced to a liquid culturing medium; US Pub. Pat. App. No. US20090130704 (published May 21, 2009) discloses bubbling gas through a liquid culturing medium. Although the cited references provide an improvement over a stagnant pool of liquid culturing medium underneath a gas, these solutions also have some drawbacks.

The cited references have not adequately improved the transfer of nutrients to the microorganisms within a liquid culturing medium. First, stirring the culturing medium and/or bubbling nutrients therethrough does not result in an adequate number of microorganisms within the medium being exposed to the nutrient for absorption, since the absorption is restricted to the gas bubble and medium interface. Second, bubbles will typically form in different sizes leading to larger/smaller doses of nutrients along differently sized bubble paths. Third, the partial pressure of the gas within a bubble decreases as nutrients are transferred to the culturing medium, which limits the gas transfer to the solution. Furthermore, the relative movement between the bubble and the solution is slow and as a result, the gas transfer rate is slow. Fourth, dissolving gaseous nutrients into a liquid before delivery to a culturing medium requires unnecessary efforts and costs since a liquid solution of dissolved gaseous nutrients does not typically present the nutrients in their naturally occurring phase. Accordingly, there is still a need for methods and apparatus that improve the delivery of gaseous nutrients to a culturing medium.

In addition to nutrient transfer problems, similar problems exist for microorganisms within a liquid culturing medium that require light for cultivation (e.g., algae). Light cannot typically penetrate to the microorganisms at the bottom of the liquid culturing medium pool. Attempted solutions to the problems include placing a light source within a liquid culturing medium and/or using a transparent cultivation zone. For example: US Pub. Pat. App. No. 20090203067 (published Aug. 13, 2009) discloses a photobioreactor that changes configurations in accordance with the suns position; US Pub. Pat. App. No. 20090148931 (published Jun. 11, 2009), 20090047722 (published Feb. 19, 2009), US Pub. Pat. App. No. 20030073231 (published Apr. 17, 2003), and US Pub. Pat. App. No. 20030059932 (published Mar. 27, 2003) disclose submerging light emitters in a culturing medium; US Pub. Pat. App. No. 20090130706 (published May 21, 2009) discloses transparent photobioreactors that float a top ponds whereby less shading is present; US Pub. Pat. No. 20090068727 (published Mar. 12, 2009) discloses a photobioreactor with a shallow channel; US Pub. Pat. App. No. 20080220515 (published Sep. 11, 2008) discloses a photobioreactor with light emitters mounted within the cultivation zone; US Pub. Pat. App. No. 20070155006 (published Jul. 5, 2007) discloses a photobioreactor comprising a system of trays with optical elements that reflect and disperse light between the trays; US Pub. Pat. App. No. 20040048364 (published Mar. 11, 2004) discloses a photobioreactor comprising light permeable material; US Pub. Pat. App. No. 20030228684 (published Dec. 11, 2003) discloses a photobioreactor comprising transparent tubes wherein light is provided to a culturing medium; US Pub. Pat. App. No. 20030170884 (published Sep. 11, 2003) discloses a tubular photobioreactor with axial light source. However, the attempted solutions have not maximized light transfer since microorganisms at depths away from the light source are not exposed to light. Accordingly, there is still a need for methods and apparatus that improve the delivery of light to a culturing medium.

Finally, many microorganisms produce gaseous metabolic products (e.g., Carbon dioxide, oxygen, methane and the like) and, like the absorption of a gaseous nutrient and/or light, the release of gaseous metabolic products is accomplished primarily at the upper surface of a culturing medium pool. However, the metabolic products can be toxic to the cultured microorganism and correspondingly may limit the amount of microorganisms cultured. For example, carbon dioxide within the culturing medium can result in carbonic acid which may alter the pH of the medium whereby microorganisms cannot grow. Attempted solutions to this problem include adding a base to the culturing medium to neutralize the acid. However, adding a base can increase the microorganism culturing costs and add additional steps to the culturing process. For this reason, there is a need for methods and apparatus that improve the removal of metabolic products.

### SUMMARY OF THE INVENTION

Accordingly, it is an object of the present application to provide an apparatus and methods of use thereof for providing improved delivery of gaseous nutrients to a culturing medium in the nutrients' natural state.

It is another object of the present application to provide an apparatus and methods of use thereof for providing improved delivery of light to a culturing medium.

It is yet another object of the present application to provide an apparatus and methods of use thereof for providing superior culturing capability to those presently known.

It is yet still an object of the present application to provide an apparatus and methods of use thereof for facilitating the removal of metabolic products from a culturing medium to those presently known.

### BRIEF DESCRIPTION OF THE FIGURES

The manner in which these objectives and other desirable characteristics can be obtained is explained in the following description and attached figures in which:

FIG. 1 depicts a vertical cross-section of an apparatus 1 with a first 100 and a second 200 chamber for culturing microorganisms.

FIG. 2 depicts a top view of the second chamber 200 and first chamber 100 of FIG. 1.

FIG. 3 depicts a cross section of a nozzle 500 that may be used in the apparatus 1 of FIG. 1.

FIG. 3A depicts the nozzle 500 of FIG. 3 with directional arrows indicating the flow of culturing medium through the nozzle.

FIG. 4 depicts a vertical cross-section of the FIG. 1 apparatus 1 with directional arrows indicating culturing medium 600 circulation within the apparatus.

It is to be noted, however, that the appended figures illustrate only typical embodiments of this invention, and therefore, are not to be considered limiting of its scope, for the invention may admit to other equally effective embodiments that will be appreciated by those reasonably skilled in the relevant arts. Also, figures are not necessarily made to scale.

### DETAILED DESCRIPTION OF PREFERRED EMBODIMENTS

In general, a preferred embodiment of the present application may be an apparatus for cultivating microorganisms. The apparatus may comprise a microorganism culturing medium reservoir and a gaseous nutrient reservoir. Within the apparatus, droplets of culturing medium may be sprayed or misted through the gaseous reservoir and/or exposed to light. While exposed to the gas and/or light, the droplets suitably absorb nutrients or energy necessary for the microorganism cultivation and/or release metabolic products to maintain a favorable growth condition. After gas nutrient and/or light exposure, the droplets preferably return to the microorganism reservoir. The more specific aspects of the preferable embodiment may be viewed in the drawings.

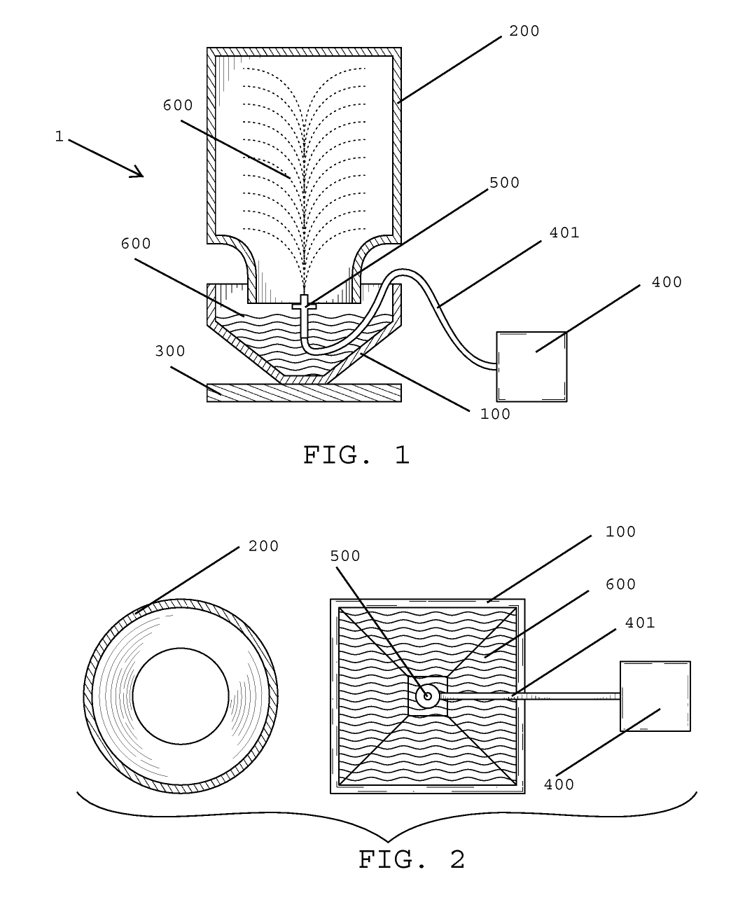

FIG. 1 is a vertical cross section of a preferable apparatus 1 for cultivating microorganisms. As can be seen in the drawing, a preferred embodiment of the apparatus 1 preferably comprises: a first chamber 100; a second chamber 200; at least one hot plate 300; at least one pump 400 with at least one exhaust hose 401; and at least one nozzle 500. A culturing medium 600 is shown within the first chamber 100.

Referring still to FIG. 1, the figure shows the apparatus 1 in an operable assembly. Suitably, the first 100 and second 200 chambers are coupled whereby the cavities of the chambers may be in fluid communication. As depicted, the coupling of the chambers is not necessarily fluid or air-tight (i.e., a gas within the gas reservoir may suitably escape from the apparatus 1 from under the second chamber 200 at the coupling). However, a fluid or air-tight coupling between the first 100 and second 200 chambers may be provided to the apparatus 1 without altering the functions or utility of the apparatus 1. Suitably, the nozzle 500 may be positioned within the culturing medium 600 and its 500 outlet tip directed toward the cavity of the second chamber 200. The pump 400 and hose 401 may be used to force air (or any gas/gas mixture) through the nozzle 500. A hot plate 300 may be used for heating the culturing medium 600.

Although a nozzle 500 connected to a pump 400 is disclosed as a delivery means for delivering culturing medium to within a second chamber 200, it is understood that a delivery means could comprise at least one pump 400 or at least one nozzle. Furthermore, it is understood that a delivery means could be a sprinkler head or device known to one of skill in the art for dispersing fluids in droplet form. Delivery means could be any other apparatus capable of forcibly ejecting or dispersing fluids, including but not limited to pneumatic or mechanical devices.

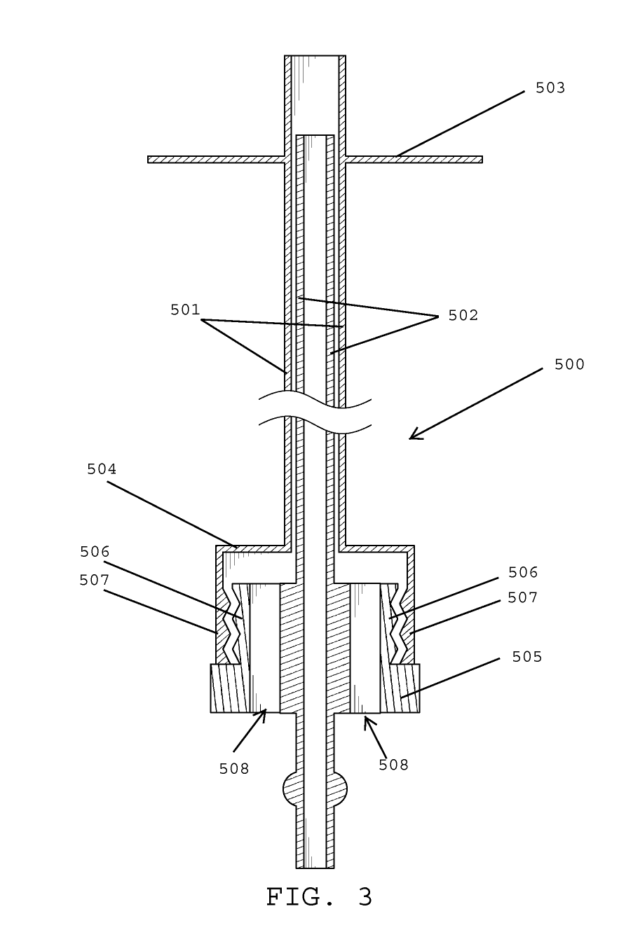

FIG. 2 depicts two views: a top view of the first chamber 100; and a top view of the second chamber 200. Preferably, the culturing medium 600 may be provided to the cavity of the first chamber 100 while the first 100 and second 200 chambers are disassociated from one-another. However, a culturing medium 600 may also be provided to the first chamber 100 while the apparatus 1 is assembled as depicted in FIG. 1. It should also be noted that, although the first 100 and second 200 chambers are drawn in specific shapes, volumetric proportions, and apparent sizes, it is contemplated by the inventor that any shape, volumetric proportion, and size could be used to create another embodiment of this application without altering the functions or results of the apparatus 1. For example: the apparatus 1 may be operated without the second chamber wherein culturing medium drops are sprayed into the atmosphere in the manner of a fountain. In addition, it is contemplated that the first and second chambers may be constructed as a single unit.

The interaction between the nozzle 500 and culturing medium 600 may be seen by referring simultaneously to FIGS. 1 and 2. As depicted, the nozzle 500 preferably is situated atop the culturing medium 600 with the hose 401 running through the medium 600 for communication to the pump 400. Although the drawings depict the hose 401 passing over a side of the first chamber 100, it is also contemplated that the hose 401 may pass through a side or bottom of the first chamber without affecting the utility of the apparatus 1.

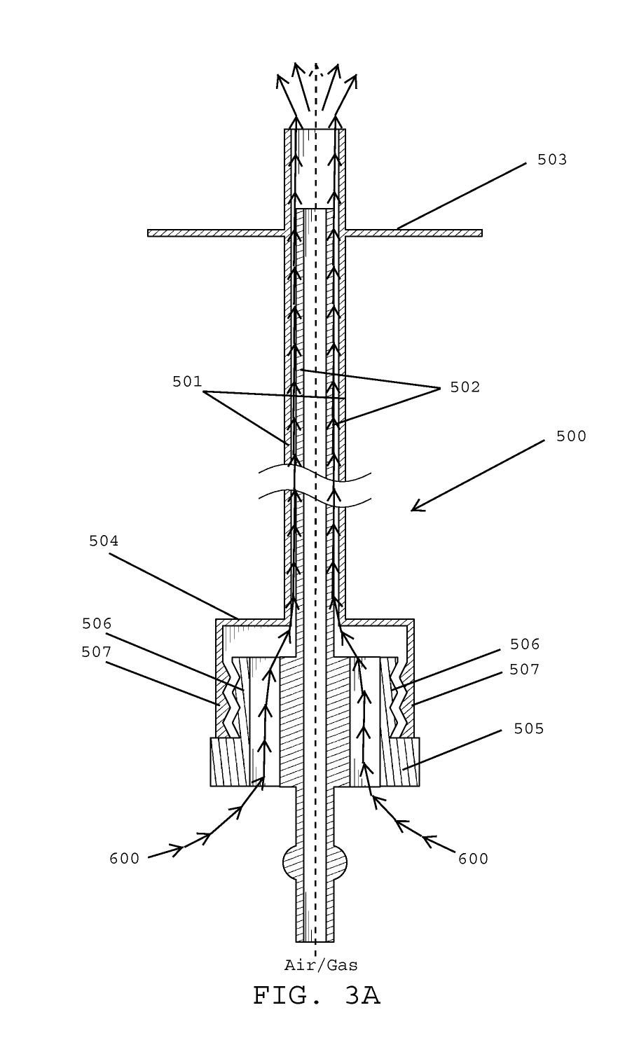

FIGS. 3 and 3A depict an embodiment of the nozzle 500 for use with the apparatus 1 depicted in the earlier figures. In general, the nozzle 500 may operate to spray or mist droplets of the medium 600 from the first chamber 100 into the second chamber 200. Suitably, the spray or mist is accomplished via forcing air through the inner tube 502 in order to correspondingly draw the culturing medium 600 through the outer tube 501 for dispersion into the second chamber 200 along with the forced air. Operably, the gas-flow in the inner tube 502 generates a negative pressure at the tip of the outer tube 501, which negative pressure forcibly draws the medium 600 through the passages 508 and outer tube 501 to spray the medium into the second chamber 200. In this manner, the culturing medium is rapidly exposed to the chosen environment of the second chamber 200. It is understood that a negative pressure could, in another embodiment, be created via forcing air in between the inner 502 and outer 501 tube whereby the culturing medium 600 is drawn through the inner tube 501 and sprayed into the second chamber 200.

As further seen in FIGS. 3 and 3A, the nozzle 500 may feature: an outer tube 501; an inner tube 502; and an optional float pad 503. The inner 502 and outer 501 tubes may connect at a lower portion 504 of the outer tube 501 and a central portion 505 of the inner tube 502. Suitably, the connection may be accomplished as depicted using male-to-female cooperating screw threads (506,507), although other connection mechanisms may be used. The central portion 505 should preferably be provided with passages 508 for the liquid culturing medium 600 to enter the outer tube 501 through the lower portion 504. The inner tube 502 will typically extend within the outer tube 501 and terminate at an upper portion 507 thereof. Although not depicted in FIG. 3, a hose 401 may connect to the inner tube 502.

The float pad 503 may be useful for positioning and orienting the nozzle. Referring back to FIGS. 1 and 2, the float pad may support the nozzle 500 tip above the culturing medium 600 whereby the nozzle 500 tip is directed toward the second chamber 200. However, while the float pad 503 may be a preferable feature, it is contemplated that the nozzle 500 does not need to float atop the culturing medium 600 and may instead be stationary or affixed to the apparatus 1.

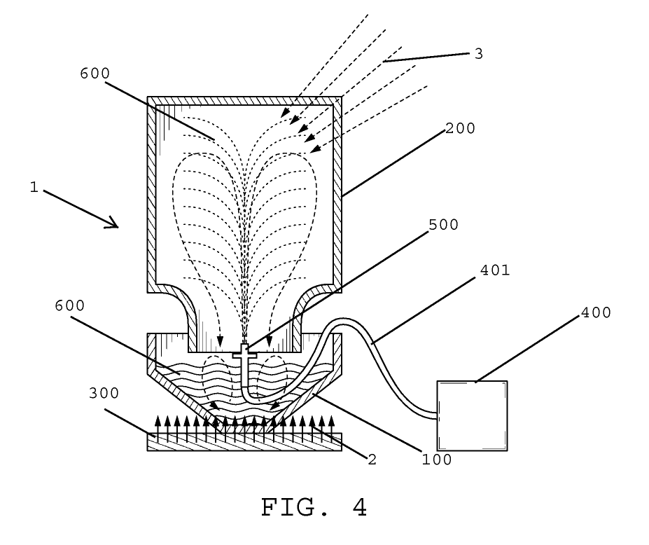

FIG. 4 shows the apparatus 1 in operation. At the bottom of the figure, the hot plate 300 may optionally be used to provide energy (heat) 2 to the medium. Heating the medium to a temperature above the ambient may be useful for circulating the medium 600 to avoid stagnation. Circulation is likely the result of the hotter portions of the medium rising within the first chamber 100. In addition, heat may be necessary for cultivating the microorganisms.

Still referring to FIG. 4, gaseous nutrients may be provided to the medium 600 via spraying or misting the medium 600 to within the second chamber 200. In moving droplet form, the cumulative surface area of the medium 600 is increased thereby exposing more microorganisms to the gaseous nutrient or any other characteristics in the chambers. Unlike with bubble-up apparatuses, the gas nutrient in the present apparatus 1 does not typically lose partial pressure and, as the droplets move through the gas, nutrients may be absorbed in higher quantities. Carbon dioxide releasing microorganisms may also benefit from an increased carbon dioxide release rate for reasons conversely related to increased nutrient absorption. It should be noted that gases of choice or other environments of choice may be introduced via the pump or by other means (e.g., filling the second chamber 200 manually).

Referring back to FIGS. 3 and 3A, if the apparatus 1 is in a vertical configuration the sprayed or misted medium may ultimately fall into the reservoir of culturing medium 600 within the first chamber 100. For example, the medium 600 may be sprayed onto the interior of the second chamber 200 whereafter the medium 600 may trickle down the chamber 200 to the reservoir. The gas may also be absorbed while the medium 600 is trickling down the sidewall.

FIG. 4 also shows light 3 penetrating a wall of the second chamber 200. Light 3 may be important for microorganisms that photosynthesize. This feature may be accomplished via fabrication of the second chamber 200 using transparent materials. It should be noted, however, that transparency is not a requirement and the second chamber 200 may optionally be opaque. Additionally, it is not necessary that the light source be outside of the second chamber 200. Rather, a light may be installed therein.

As an improvement over prior art apparatuses with a light-source within the culturing medium, the present apparatus 1 may provide light to a medium 600 at a higher rate. The increased exposure to light may be due to the penetration of the light through the droplets. Unlike a pooled culturing medium 600, droplets are small enough for the light to completely penetrate and, accordingly, a higher percentage of microorganisms within the droplet are exposed.

Example: In one non-limiting example, the apparatus 1 depicted by the figures has been used to culture a BL21(DE3) strain of E. coli bacteria at ambient room temperature. A liter of culturing medium was created using a Luria-Bertani (LB) broth containing 10 grams of tryptone, 5 grams of yeast extract, and 10 grams of sodium chloride. The identified culturing medium (400 milli-liters) was placed in the apparatus 1, inoculated with a small culture (<40 micro-liters) of E. coli strain, BL21(DE3), and circulated according to the above disclosure. The E. coli reached stationary phase after twelve (12) hours. For comparison, E. coli cultivated using a typical stir-bubble cultivating apparatus and similar amounts of the identified ingredients took 15 hours to reach a stationary phase. Thus, the apparatus and methods disclosed herein have resulted in significant improvements to culturing technologies.

The present application provides a number of benefits over the prior art. First, providing the culturing medium in droplet form to a gas nutrient reservoir increases the surface area of the culturing medium which can be translated to a higher nutrient-to-medium transfer rate. Second, the culturing medium, in droplet form, moves through the gas nutrient at speeds that are faster than air bubbles rising through a liquid culturing medium pool, thereby further resulting in higher nutrient-to-medium transfer rates. Third, gaseous nutrients bubbling through a culturing medium pool have lower nutrient to medium transfer rates since the gas bubbles lose pressure. Conversely, droplets of culturing medium moving through a gas reservoir have constant nutrient transfer rates. Fourth, culturing medium droplets have a better light transmission than a culturing medium pool. Finally, metabolic products are released by the microorganisms in droplet form at a higher rate than within a liquid culturing medium pool. All of these advancements, among others, illustrate the improvement in nutrient delivery, photosynthesis, and the release of metabolic waste.

It should be noted that FIGS. 1 through 4 and the associated description are of illustrative importance only. In other words, the depiction and descriptions of the present invention should not be construed as limiting of the subject matter in this application. The apparatus and methods discussed hereby are susceptible to modification without changing the overall concept of the disclosure. For example, another embodiment of the present disclosure features, as a culture medium delivery means, multiple nozzles for spraying culturing medium to within a second chamber simultaneously. Yet another embodiment may feature a delivery means disposed at the top of the second chamber with the culturing medium sprayed, misted, or dripped downward. Still another embodiment may feature the first and second chambers in horizontal alignment with culturing medium sprayed/misted horizontally. In yet another embodiment, the apparatus may contain at least two nozzles: one for spraying a gaseous nutrient into a second chamber; and one for spraying the culturing medium into the second chamber. Additional modifications may become apparent to one skilled in the art after reading this disclosure.

I claim:

1. An apparatus comprising:
a delivery means for delivering at least one droplet of culturing medium to a gaseous nutrient.

2. The apparatus of claim 1 further comprising:
a first chamber for housing the culturing medium;
a second chamber for housing the gaseous nutrient; and,
a pump for spraying said droplet(s) into the gaseous nutrient via said delivery means.

3. The apparatus of claim 2 wherein the delivery means is a nozzle connected to the pump.

4. The apparatus of claim 3 wherein the nozzle comprises:
an inner tube;
an outer tube around the inner tube, said inner tube terminating short of the outer tube; and,
at least one passage.

5. The apparatus of claim 4 wherein said nozzle further comprises a float pad.

6. A method of culturing a microorganism comprising the step of
locating a gaseous nutrient; and,
delivering at least one droplet of culturing medium to the gaseous nutrient.

7. The method of claim 6 further comprising the steps of:
obtaining an apparatus comprising a nozzle, a first chamber, and a second chamber;
obtaining a reservoir of the culturing medium and locating the same in the first chamber;
providing the gaseous nutrient to the second chamber; and,
delivering said droplet(s) of the culturing medium to the gaseous nutrient via the nozzle.

8. The method of claim 7 further comprising the step of trickling the droplet(s) down a wall of the second chamber.

9. The method of claim 7 further comprising the step of collecting the droplet(s) in the first chamber

10. The method of claim 8 further comprising the step of forcing the gaseous nutrient through the nozzle.

11. The method of claim 8 wherein the apparatus further comprises:
a pump for forcing the gaseous nutrient through the nozzle.

12. The method of claim 7, wherein the nozzle comprises:
an inner tube;
an outer tube around the inner tube, said inner tube terminating short of the outer tube; and,
at least one passage.

13. The method of claim 12 wherein said step of forcing air through a nozzle is preceded by the step of forcing air into said inner tube.

14. The method of claim 13 further comprising the step of drawing the culturing medium into the outer tube via the passage.

15. The method of claim 14 wherein said step of forcing air through a nozzle is preceded by the step of forcing air in between said outer and inner tube.

16. The method of claim 6 wherein:
the microorganism is E. coli; and,
the culturing medium is a solution of Lauria-Bertani broth initially containing tryptone, yeast extract, and sodium chloride.

17. The method of claim 6 wherein the step of delivering at least one droplet of culturing medium to the gaseous nutrient is accomplished via generating a negative pressure at a tip of a nozzle whereby the culturing medium is forcibly sprayed from the tip of the nozzle.

18. The method of claim 6 further comprising the steps of:
providing a light source; and,
exposing the droplet(s) of the culturing medium to the light source.

19. The method of claim 6 further comprising the step of:
Releasing metabolic products from the droplet(s) to within the gas nutrient.

20. A method for removing metabolic products from microorganism cultures comprising the steps of:
identifying a culturing medium; and
delivering at least one droplet of the culturing medium to a gaseous reservoir.
